(19) United States
(12) Patent Application Publication
(10) Pub. No.: US 2004/0224965 A1
(43) Pub. Date: Nov. 11, 2004

(54) 2-Guanidino-4-arylchinazolines as nhe-3 inhibitors

(76) Inventors: Rolf Gericke, Seeheim (DE); Norbert Beier, Reinheim (DE); Claudia Wilm, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/257,636

(22) PCT Filed: Mar. 22, 2001
(86) PCT No.: PCT/EP01/03281

(30) Foreign Application Priority Data
Apr. 18, 2000 (DE) 100 19 062.6

(51) Int. Cl. A61K 31/517; C07D 239/84

(52) U.S. Cl. 514/266.4; 544/292

(57) ABSTRACT

The invention relates to compounds of formula (I), where Ar=unsubstituted, or monosubstituted by R³, phenyl, or naphthyl; R¹, R²=independently, A, OA, Hal or CF₃; R³=A, OA, Hal, or CF₃; A=1-6C alkyl and Hal=F, Cl, Br or I and the salts and solvates thereof as NHE3 inhibitors.

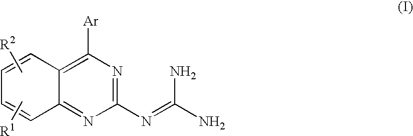

The invention relates to compounds of the formula I

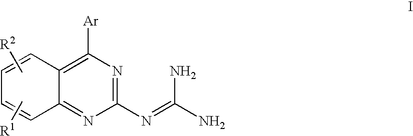

in which

Ar is unsubstituted or mono-R³-substituted phenyl or naphthyl,

R¹ and R² are each, independently of one another, H, A, OA, Hal or CF₃,

R³ is A, OA, Hal or CF₃,

A is alkyl having 1, 2, 3, 4, 5 or 6 carbon atoms, and

Hal is F, Cl, Br or I,

and their physiologically acceptable salts and solvates as NHE-3 inhibitors.

The formula I also covers the tautomeric compounds of the formula I′

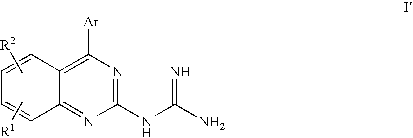

Other inhibitors of the sodium/proton exchanger subtype 3 are described, for example, in EP 0 825 178.

The compounds of the formulae I and I′ have already been described in U.S. Pat. No. 3,131,187, as has their use for other purposes.

Quinazolinylguanidine derivatives have been described by V. I. Shvedov et al. in Pharm. Chem. J. (Engl. transl.) 1980, 14, 532-538 or in Khim. Farm. Zh. 1980, 14, 38-43, and by S. C. Bell et al. in J. Med. Pharm. Chem. 1962, 5, 6369.

The invention had the object of finding novel compounds having valuable properties, in particular those which can be used for the preparation of medicaments:

Surprisingly, it has been found that the compounds of the formula I and their salts are well tolerated and inhibit sodium/proton exchanger subtype 3.

The compounds of the formula I can be employed as medicament active ingredients in human and veterinary medicine.

It is known that the Na⁺/H⁺ exchanger represents a family having at least six different isoforms (NHE-1 to NHE-6), all of which have now been cloned. While subtype NHE-1 is distributed ubiquitously in all tissues throughout the body, the other NHE subtypes are expressed selectively in specific organs, such as in the kidney or in the lumen wall and contraluminal wall of the small intestine. This distribution reflects the specific functions that the various isoforms serve, namely on the one hand regulation of the intracellular pH and cell volume by subtype NHE-1 and on the other hand Na⁺ absorption and resorption in the intestine and kidney by isoforms NHE-2 and NHE-3. Isoform NHE-4 has been found principally in the stomach. Expression of NHE-5 is restricted to the brain and neuronal tissue. NHE-6 is the isoform that forms the sodium/proton exchanger in the mitochondria.

Isoform NHE-3 is expressed in particular in the apical membrane of the proximal renal tubuli; an NHE-3 inhibitor therefore-exerts, inter alia, a protective action on the kidneys.

The therapeutic use of a selective inhibitor for NHE-3 isoforms is manifold. NHE-3 inhibitors inhibit or reduce tissue damage and cell necrosis after pathophysiological hypoxic and ischemic events which result in activation of the NHE activity, as is the case during renal ischemia or during the removal, transport and reperfusion of a kidney during a kidney transplant.

The compounds of the formula I have a cytoprotective action in that they prevent the excessive absorption of sodium and water into the cells of organs undersupplied with oxygen.

The compounds of the formula I have a hypotensive action and are suitable as medicament active ingredients for the treatment of hypertonia. They are furthermore suitable as diuretics.

The compounds of the formula I, alone or in combination with NHE inhibitors of other subtype specificity, have an antiischemic action and can be used in the case of thromboses, atherosclerosis, vascular spasms, for the protection of organs, for example kidney and liver, before and during operations, and in the case of chronic or acute renal failure.

They can furthermore be used for the treatment of strokes, cerebral oedema, ischemia of the nervous system, various forms of shock, for example allergic, cardiological, hypovolaeic or bacterial shock, and for improving breathing drive in, for example, the following states: central sleep apnea, cot death, postoperative hypoxia and other breathing disorders.

Through combination with a carboanhydrase inhibitor, breathing activity can be further improved.

The compounds of the formula I have an inhibiting effect on the proliferation of cells, for example fibroblast cell proliferation and the proliferation of the smooth muscle cells, and can therefore be used for the treatment of illnesses in which cell proliferation is a primary or secondary cause.

The compounds of the formula I can be used against delayed complications of diabetes, cancer illnesses, fibrotic illnesses, endothelial dysfunction, organ hypertrophia and hyperplasia, in particular in prostate hyperplasia or prostate hypertrophia.

They are furthermore suitable as diagnostic agents for the determination and differentiation of certain forms of hypertonia, atherosclerosis, diabetes and proliferative illnesses.

Since the compounds of the formula I also have an advantageous effect on the level of serum lipoproteins, they can be employed, alone or in combination with other medicaments, for the treatment of an increased blood fat level.

The invention relates to the use of compounds of the formula I according to claim 1 and their physiologically acceptable salts and/or solvates for the preparation of a medicament for the treatment of thrombosis, ischemic states of the heart, of the peripheral and central nervous system and of strokes, ischemic states of peripheral organs and extremities and for the treatment of shock states.

The invention furthermore relates to the use of compounds of the formula I according to claim 1 and their physiologically acceptable salts and/or solvates for the preparation of a medicament for use in surgical operations and organ transplants and for the preservation and storage of transplants for surgical measures.

The invention also relates to the use of compounds of the formula I according to claim 1 and their physiologically acceptable salts and/or solvates for the preparation of a medicament for the treatment of illnesses in which cell proliferation is a primary or secondary cause, for the treatment or prophylaxis of disorders of fat metabolism or disturbed breathing drive.

The invention furthermore relates to the use of compounds of the formula I according to claim 1 and their physiologically acceptable salts and/or solvates for the preparation of a medicament for the treatment of renal ischemia, ischemic intestinal illnesses or for the prophylaxis of acute or chronic renal illnesses.

Methods for the identification of substances which inhibit sodium/proton exchanger subtype 3 are described, for example, in U.S. Pat. No. 5,871,919.

For all radicals in the compounds of the formula I which occur more than once, such as, for example, A, their meanings are independent of one another.

The term hydrates and solvates is taken to mean, for example, the hemi-, mono- or dihydrates, and the term solvates is taken to mean, for example, alcohol addition compounds, such as, for example, with methanol or ethanol.

In the formulae above, A is alkyl, is linear or branched, and has 1, 2, 3, 4, 5 or 6 carbon atoms. A is preferably methyl, furthermore ethyl, propyl, iso-propyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, or 1,1,2- or 1,2,2-trimethylpropyl.

OA is preferably methoxy, ethoxy, propoxy, isopropoxy or butoxy.

Hal is preferably F, Cl or Br, but also I.

Ar is preferably unsubstituted phenyl or naphthyl, furthermore preferably phenyl or naphthyl which is monosubstituted, for example, by A, fluorine, chlorine, bromine, iodine, methoxy, ethoxy, propoxy, butoxy or CF₃.

Accordingly, the invention relates in particular to the use of the compounds of the formula I in which at least one of the said radicals has one of the preferred meanings indicated above. Some preferred groups of compounds may be expressed by the following sub-formulae Ia to II, which conform to the formula I and in which the radicals not designated in greater detail have the meaning indicated in the formula I, but in which

| | | |
|---|---|---|
| in Ia | R¹ | is H or Hal; |
| in Ib | R¹ | is H or Hal, |
| | R² | is H; |
| in Ic | R¹ | is H or Hal, |
| | R² | is H |
| | Ar | is phenyl; |
| in Id | R¹ | is H or Hal, |
| | R² | is H |
| | R³ | is A, OA or Hal; |
| in Ie | Ar | is phenyl; |
| in If | Ar | is phenyl, |
| | R^{1 and R}² | are each, independently of one another, H, A, OA, |
| | | Hal or CF₃; |
| in Ig | Ar | is unsubstituted or mono-R³-substituted phenyl, |
| | R¹ | is H or Hal, |
| | R² | is H |
| | R³ | is A, OA or Hal; |
| in Ih | Ar | is mono-R³-substituted phenyl, |
| | R¹ | is H or Hal, |
| | R² | is H, |
| | R³ | is A, OA or Hal; |
| in Ii | Ar | is mono-R³-substituted phenyl, |
| | R¹ | is H, Hal, OA or A, |
| | R² | is H, |
| | R³ | is Hal; |
| in Ij | Ar | is mono-R³-substituted phenyl, |
| | R¹ | is H, Hal, OA or A, |
| | R² | is H or OA, |
| | R³ | is Hal; |
| in Ik | Ar | is unsubstituted or mono-R³-substituted phenyl, |
| | R¹ | is H, Hal, OA or A, |
| | R² | is H or OA, |
| | R³ | is Hal; |
| in Il | Ar | is unsubstituted or mono- or di-R³-substituted phenyl, |
| | R¹ | is H, Hal, OA or A, |
| | R² | is H, Hal, OA or A, |
| | R³ | is Hal or A, |
| | A | is alkyl having 1, 2, 3 or 4 carbon atoms or CF₃. |

The invention also relates to the novel compounds selected from the group consisting of

6-chloro-4-(2-fluorophenyl)-2-quinazolinylguanidine,

6-bromo-4-(2-fluorophenyl)-2-quinazolinylguanidine,

6,7-dimethoxy-4-phenyl-2-quinazolinylguanidine,

7-chloro-4-(2-fluorophenyl)-2-quinazolinylguanidine,

6-chloro-4-(4-methylphenyl)-2-quinazolinylguanidine,

8-methyl-4-phenyl-2-quinazolinylguanidine,

6-chloro-4-(2-methylphenyl)-2-quinazolinylguanidine,

6-chloro-4-(4-ethylphenyl)-2-quinazolinylguanidine,

6-trifluoromethyl-4-phenyl-2-quinazolinylguanidine,

6-chloro-4-(3,4-dimethylphenyl)-2-quinazolinylguanidine,

6-chloro-4-(3-fluoro-4-methylphenyl)-2-quinazolinylguanidine,

6-chloro-4-(3-chloro-4-methylphenyl)-2-quinazolinylguanidine,

6-chloro-4-(4-ethylphenyl)-2-quinazolinylguanidine,

6-chloro-4-(4-trifluoromethylphenyl)-2-quinazolinylguanidine,

6-chloro-8-fluoro-4-(4-methylphenyl)-2-quinazolinylguanidine,

6-chloro-7-methyl-4-(4-methylphenyl)-2-quinazolinylguanidine,

6-chloro-4-(2,4-dimethylphenyl)-2-quinazolinylguanidine,

6-chloro-4-(3-bromophenyl)-2-quinazolinylguanidine,

6-chloro-4-(4-bromophenyl)-2-quinazolinylguanidine,

6-chloro-4-(4-isopropylphenyl)-2-quinazolinylguanidine,

6-chloro-4-(2-bromophenyl)-2-quinazolinylguanidine,

6-chloro-4-(3-fluoro-4-trifluoromethylphenyl)-2-quinazolinylguanidine,

6-chloro-8-methyl-4-(4-methylphenyl)-2-quinazolinylguanidine,

6-chloro-4-(4-fluorophenyl)-2-quinazolinylguanidine,

6-chloro-4-(2-chlorophenyl)-2-quinazolinylguanidine,

4-(3-methylphenyl)-2-quinazolinylguanidine,

6-chloro-4-(3-fluorophenyl)-2-quinazolinylguanidine,

6-chloro-8-chloro-4-phenyl-2-quinazolinylguanidine,

6-chloro-7-chloro-4-phenyl-2-quinazolinylguanidine,

and their physiologically acceptable salts and solvates.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

The starting materials can, if desired, also be formed in situ, so that they are not isolated from the reaction mixture, but instead are immediately converted further into the compounds of the formula I.

The 2-guanidino-4-arylquinazolines of the formula I are preferably prepared by reacting o-aminophenyl ketones of the formula II

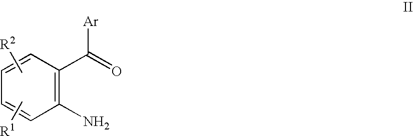

in which R¹, R² and Ar are as defined in claim 1, with 1-cyanoguanidine.

The reaction is carried out in an inert solvent.

Examples of suitable inert solvents are hydrocarbons, such as hexane, petroleum ether, benzene, toluene or xylene; chlorinated hydrocarbons, such as tricloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

DMF, water or an alcohol is preferably used.

The reaction is very particularly preferably carried out without a solvent, i.e. in the melt, at temperatures between 100 and 200° C.

Of advantage is the presence of an acidic catalyst, such as AlCl₃, TiCl₄, p-toluenesulfonic acid, BF₃, acetic acid, sulfuric acid, oxalic acid, POCl₃ or phosphorus pentoxide.

A preferred variant comprises employing one of the reactants already as a salt, for example as the hydrochloride.

A further valuable method for the preparation of the compounds of the formula I comprises reacting, instead of 1-cyanoguanidine, a compound of the formula II

HN═CX—NH—C(═NH)—NH₂ III

in which

X is —SA, —SAr, OA or OAr

and Ar and A are, for example, as defined in claim 1,

with a compound of the formula II.

Finally, the compounds of the formula I can be prepared by reaction of 2-chloro-4-arylquinazolines of the formula IV

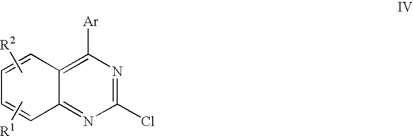

in which Ar, R¹ and R² are as defined in claim 1,

with guanidine.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and disulfonic acids, and laurylsulfuric acid. Salts with physiologically unacceptable acids, for example picrates, can be used for the isolation and/or purification of the compounds of the formula I.

The invention furthermore relates to the use of the compounds of the formula I as NHE-3 inhibitors and/or their physiologically acceptable salts for the preparation of pharmaceutical preparations, in particular by non-chemical methods. In this case, they can be converted into a suitable dosage form together with at least one solid, liquid and/or semiliquid excipient or assistant, and, if desired, in combination with one or more further active ingredients.

The invention furthermore relates to pharmaceutical preparations comprising at least one NHE-3 inhibitor of the formula I and/or one of its physiologically acceptable salts and solvates.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders, or transdermally in patches.

The novel compounds may also be lyophilized and the resultant lyophilisates used, for example, for the preparation of injection preparations. The preparations indicated may be sterilized and/or comprise assistants, such as lubricants, preservatives, stabilizers-and/or wetting agents, emulsifiers, salts for modifying the osmotic pressure, buffer substances, colorants and flavours and/or a plurality of further active ingredients, for example one or more vitamins.

Suitable pharmaceutical preparations for administration in the form of aerosols or sprays are, for example, solutions, suspensions or emulsions of the active ingredient of the formula I in a pharmaceutically acceptable solvent.

The compounds of the formula I and their physiologically acceptable salts and solvates can be used for the treatment and/or prophylaxis of the illnesses or illness states described above.

In general, the substances according to the invention are preferably administered in doses between about 0.1 and 500 mg, in particular between 1 and 10 mg, per dosage unit. The daily dose is preferably between about 0.001 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

### EXAMPLES

Preferred NHE-3 inhibitors are the compounds selected from the group consisting of

4-phenyl-2-quinazolinylguanidine, m.p. 247-250° C. (decomposition;

4-phenyl-2-quinazolinylguanidine, hydrochloride, m.p. 236-238° C.;

6-chloro-4-phenyl-2-quinazolinylguanidine,

6-chloro-4-phenyl-2-quinazolinylguanidine, hydrochloride, m.p. 309-310° C.;

4-(4-bromophenyl)-2-quinazolinylguanidine, hydrochloride, m.p. 185-189° C.;

4-(4-chlorophenyl)-2-quinazolinylguanidine, hydrochloride, m.p. 296-297° C.;

4-(4-methoxyphenyl)-2-quinazolinylguanidine, hydrochloride, m.p. 275-277° C.;

4-(4-methyl phenyl)-2-quinazolinylguanidine, hydrochloride, m.p. 300-301° C.;

6-chloro-4-(2-fluorophenyl)-2-quinazolinylguanidine, hydrochloride, m.p. 275-276°;

7-methyl-4-phenyl-2-quinazolinylguanidine, hydrochloride, m.p. 300-301° C.;

6-bromo-4-(2-fluorophenyl)-2-quinazolinylguanidine, hydrochloride, m.p. 294-295°;

7-chloro-4-phenyl-2-quinazolinylguanidine, hydrochloride, m.p. 288-290° C.;

7-methoxy-4-phenyl-2-quinazolinylguanidine, hydrochloride, m.p. 280-282° C.;

5-methoxy-4-phenyl-2-quinazolinylguanidine, hydrochloride, m.p. 272-273° C.;

6,7-dimethoxy-4-phenyl-2-quinazolinylguanidine, hydrochloride, m.p. 220-222° C.;

6-methoxy-4-phenyl-2-quinazolinylguanidine, hydrochloride, m.p. 278-279° C.;

8-chloro-4-phenyl-2-quinazolinylguanidine, hydrochloride, m.p. 309-310° C.;

5-chloro-4-phenyl-2-quinazolinylguanidine, hydrochloride, m.p. 300° C.;

7-chloro-4-(2-fluorophenyl)-2-quinazolinylguanidine, hydrochloride, m.p. 281-283° C.;

6-chloro-4-(4-chlorophenyl)-2-quinazolinylguanidine, hydrochloride, m.p. 261-262° C.;

6-bromo-4-phenyl-2-quinazolinylguanidine, hydrochloride, decomp. 291-293° C.;

6-methyl-4-phenyl-2-quinazolinylguanidine, hydrochloride, m.p. 295-296° C.;

6-fluoro-4-phenyl-2-quinazolinylguanidine, hydrochloride, m.p. 283-285° C.;

6-fluoro-4-(4-methylphenyl)-2-quinazolinylguanidine, hydrochloride, m.p. 193-195° C.;

6-chloro-4-(4-methylphenyl)-2-quinazolinylguanidine, hydrochloride, m.p. 312° C.;

8-methyl-4-phenyl-2-quinazolinylguanidine, hydrochloride, m.p. 285-286° C.;

6-chloro-4-(2-methylphenyl)-2-quinazolinylguanidine, hydrochloride, m.p. 308° C.;

6-chloro-4-(4-methylphenyl)-2-quinazolinylguanidine, hydrochloride, m.p. 336° C.;

6-trifluoromethyl-4-phenyl-2-quinazolinylguanidine, hydrochloride, m.p. 300-302° C.;

6-chloro-4-(3,4-dimethylphenyl)-2-quinazolinylguanidine, hydrochloride, m.p. 323-325° C.;

6-chloro-4-(3-fluoro-4-methylphenyl)-2-quinazolinylguanidine, hydrochloride, m.p. 317-320° C.;

6-chloro-4-(3-chloro-4-methylphenyl)-2-quinazolinylguanidine, hydrochloride, m.p. 336-338° C.;

6-chloro-4-(4-ethylphenyl)-2-quinazolinylguanidine, p-toluenesulfonate, m.p. 179-184° C.;

6-chloro-4-(4-trifluoromethylphenyl)-2-quinazolinylguanidine, dihydrochloride, m.p. 329-332° C.;

6-chloro-8-fluoro-4-(4-methylphenyl)-2-quinazolinylguanidine, p-toluenesulfonate, m.p. 290-300° C.;

6-chloro-7-methyl-4-(4-methylphenyl)-2-quinazolinylguanidine, p-toluenesulfonate, m.p. 360° C.;

6-chloro-4-(2,4-dimethylphenyl)-2-quinazolinylguanidine, p-toluenesulfonate;

6-chloro-4-(3-bromophenyl)-2-quinazolinylguanidine, hydrochloride, m.p. 319-323° C.;

6-chloro-4-(4-bromophenyl)-2-quinazolinylguanidine, hydrochloride, m.p. 330° C.;

6-chloro-4-(4-isopropylphenyl)-2-quinazolinylguanidine, hydrochloride, m.p. 326-329° C.;

6-chloro-4-(2-bromophenyl)-2-quinazolinylguanidine, hydrochloride, m.p. 316-318° C.;

6-chloro-4-(3-fluoro-4-trifluoromethylphenyl)-2-quinazolinylguanidine, hydrochloride, m.p. 230-232° C.;

6-chloro-8-methyl-4-(4-methylphenyl)-2-quinazolinylguanidine, hydrochloride, m.p. 310° C.;

6-chloro-4-(4-fluorophenyl)-2-quinazolinylguanidine, hydrochloride, m.p. 346-348° C.;

6-chloro-4-(2-chlorophenyl)-2-quinazolinylguanidine, p-toluenesulfonate, m.p. 332-336° C.;

4-(3-methylphenyl)-2-quinazolinylguanidine, hydrochloride, m.p. 160-163° C.;

6-chloro-4-(3-fluorophenyl)-2-quinazolinylguanidine, hydrochloride, decomposition from 308° C.;

6-chloro-8-chloro-4-phenyl-2-quinazolinylguanidine, hydrochloride, m.p. 163-166° C.;

6-chloro-7-chloro-4-phenyl-2-quinazolinylguanidine, p-toluenesulfonate, m.p. 269-271° C.

Pharmacological Tests

The method used for the characterization of the compounds of the formula I as NHE-3 inhibitors is described below.

The compounds of the formula I were characterized with respect to their selectivity for the NHE-1 to NHE-3 isoforms. The three isoforms were expressed in stable form in mouse fibroblast cell lines. The inhibitory action of the compounds was assessed by determination of the EIPA-sensitive take-up of ²²Na⁺ into the cells after intracellular acidosis.

Material and Methods

LAP1 cell lines which express the different NHE isoforms The LAP1 cell lines which express the NHE-1, -2 and -3 isoforms (a mouse fibroblast cell line) was obtained from Prof. J. Pouysségur (Nice, France). The transfection was carried out by the method of Franchi et al. (1986). The cells were cultivated in Dulbeccos modified eagle medium (DMEM) with 10% of deactivated foetal calf serum (FCS). For selection of the NHE-expressing cells, the so-called “acid killing method” of Sardet et al. (1989) was used. The cells were firstly incubated for 30 minutes in an NH₄Cl-containing bicarbonate- and sodium-free buffer. The extracellular NH₄Cl was then removed by washing with a bicarbonate-, NH₄Cl- and sodium-free buffer. The cells Were subsequently incubated in a bicarbonate-free NaCl-containing buffer. Only those cells which functionally express NHE were able to survive in the intracellular acidification to which they were subjected.

Characterization of NHE Inhibitors with Respect to their Isoform Selectivity

With the above-mentioned mouse fibroblast cell lines which express the NHE-1, NHE-2 and NHE-3 isoforms, compounds were tested for selectivity with respect to the isoforms by the procedure described by Counillon et al. (1993) and Scholz et al. (1995). The cells were acidified intracellularly by the NH₄Cl prepulse method and subsequently by incubation in a bicarbonate-free 22Na⁺-containing buffer. Owing to the intracellular acidification, NHE was activated, and sodium was taken up into the cells. The effect of the test compound was expressed as inhibition of EIPA (ethylisopropylamiloride)-sensitive ²²Na⁺ take-up.

The cells which expressed NHE-1, NHE-2 and NHE-3 were sown out in a density of 5-7.5×10⁴ cells/well in 24-well microtitre plates and cultured to confluence for from 24 to 48 hours. The medium was removed-by suction, and the cells were incubated for 60 minutes at 37° C. in NH₄Cl buffer (50 mM NH₄Cl, 70 mM choline chloride, 15 mM MOPS, pH 7.0). The buffer was subsequently removed, and the cells were rapidly covered twice with the choline chloride wash buffer (120 mM choline chloride, 15 mM PIPES/tris, 0.1 mM ouabain, 1 mM MgCl₂, 2 mM CaCl₂, pH 7.4); the cells were incubated in this buffer for 6 minutes. After expiry of the incubation time, the incubation buffer was removed by suction. In order to remove extra-cellular radioactivity, the cells were washed rapidly four times with ice-cold phosphate-buffered saline solution (PBS). The cells were then solubilized by addition of 0.3 ml of 0.1 N NaOH per well. The cell fragment-containing solutions were transferred into scintillation tubes. Each well was then washed twice with 0.3 ml of 0.1 N NaOH, and the washing solutions were likewise introduced into the corresponding scintillation tubes. Scintillation cocktail was added to the tubes containing the cell lysate, and the radio-activity taken up into the cells was determined by determination of the β radiation.

Literature:

Counillon et al. (1993) Mol. Pharmacol. 44: 1041-1045

Franchi et al. (1986) Proc. Natl. Acad. Sci. USA 83: 9388-9392

Morgan and Canessa (1990) J. Membrane Biol. 118,193-214

Sardet et al. (1989) Cell 56: 271-280

Scholz et al. (1995) Cardiovasc. Res. 29: 260-268

The examples below relate to pharmaceutical preparations:

### Example A

### Injection Vials

A solution of 100 g of an NHE-3 inhibitor of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### Example B

### Suppositories

A mixture of 20 g of an NHE-3 inhibitor of the formula I is melted with 100 g of soya lecithin and 1400 g of cocoa butter, poured into moulds and allowed to cool. Each suppository contains 20 mg of active ingredient.

### Example C

### Solution

A solution is prepared from 1 g of an NHE-3 inhibitor of the formula I, 9.38 g of NaH₂PO₄ 2H₂O, 28.48 g of Na₂HPO₄.12H₂O and 0.1 g of benzalkonium chloride in 940 ml of bidistilled water. The pH is adjusted to 6.8, and the solution is made up to 1 l and sterilized by irradiation. This solution can be used in the form of eye drops.

### Example D

### Ointment

500 mg of an NHE-3 inhibitor of the formula I are mixed with 99.5 g of Vaseline under aseptic conditions.

### Example E

### Tablets

A mixture of 1 kg of an NHE-3 inhibitor of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, tragacanth and dye.

### Example G

### Capsules

2 kg of an NHE-3 inhibitor of the formula I are introduced into hard gelatine capsules in a conventional manner in such a way that each capsule contains 20 mg of the active ingredient.

### Example H

### Ampoules

A solution of 1 kg of an NHE-3 inhibitor of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 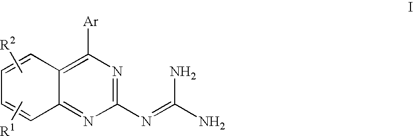
in which
Ar is unsubstituted or mono-R³-substituted phenyl or naphthyl,
R¹ and R² are each, independently of one another, H, A, OA, Hal or CF₃,
R³ is A, OA, Hal or CF₃,
A is alkyl having 1, 2, 3, 4, 5 or 6 carbon atoms, and
Hal is F, Cl, Br or I,
and their physiologically acceptable salts and-solvates as NHE-3 inhibitors.

2. Use of compounds of the formula I according to claim 1 and their physiologically acceptable salts and/or solvates for the preparation of a medicament for the treatment of hypertonia, thromboses, ischemic states of the heart, of the peripheral and central nervous system and of strokes, ischemic states of peripheral organs and extremities, and for the treatment of shock states.

3. Use of compounds of the formula I according to claim 1 and their physiologically acceptable salts and/or solvates for the preparation of a medicament for use in surgical operations and organ transplants and for the preservation and storage of transplants for surgical measures.

4. Use of compounds of the formula I according to claim 1 and their physiologically acceptable salts and/or solvates for the preparation of a medicament for the treatment of illnesses in which cell proliferation is a primary or secondary cause, for the treatment or prophylaxis of disorders of fat metabolism or disturbed breathing drive.

5. Use of compounds of the formula I according to claim 1 and their physiologically acceptable salts and/or solvates for the preparation of a medicament for the treatment of renal ischemia, ischemic intestinal illnesses or for the prophylaxis of acute or chronic renal illnesses.

6. Pharmaceutical preparation, characterized by a content of at least one NHE-3 inhibitor according to claim 1 and/or one of its physiologically acceptable salts and/or solvates.

7. Compounds selected from the group consisting of
6-chloro-4-(2-fluorophenyl)-2-quinazolinylguanidine,
6-bromo-4-(2-fluorophenyl)-2-quinazolinylguanidine,
6,7-dimethoxy-4-phenyl-2-quinazolinylguanidine,
7-chloro-4-(2-fluorophenyl)-2-quinazolinylguanidine,
6-chloro-4-(4-methylphenyl)-2-quinazolinylguanidine,
8-methyl-4-phenyl-2-quinazolinylguanidine,
6-chloro-4-(2-methylphenyl)-2-quinazolinylguanidine,
6-chloro-4-(4-methylphenyl)-2-quinazolinylguanidine,
6-trifluoromethyl-4-phenyl-2-quinazolinylguanidine,
6-chloro-4-(3,4-dimethylphenyl)-2-quinazolinylguanidine,
6-chloro-4-(3-fluoro-4-methylphenyl)-2-quinazolinylguanidine,
6-chloro-4-(3-chloro-4-methylphenyl)-2-quinazolinylguanidine,
6-chloro-4-(4-ethylphenyl)-2-quinazolinylguanidine,
6-chloro-4-(4-trifluoromethylphenyl)-2-quinazolinylguanidine,
6-chloro-8-fluoro-4-(4-methylphenyl)-2-quinazolinylguanidine,
6-chloro-7-methyl-4-(4-methylphenyl)-2-quinazolinylguanidine,
6-chloro-4-(2,4-dimethylphenyl)-2-quinazolinylguanidine,
6-chloro-4-(3-bromophenyl)-2-quinazolinylguanidine,
6-chloro-4-(4-bromophenyl)-2-quinazolinylguanidine,
6-chloro-4-(4-isopropylphenyl)-2-quinazolinylguanidine,
6-chloro-4-(2-bromophenyl)-2-quinazolinylguanidine,
6-chloro-4-(3-fluoro-4-trifluoromethylphenyl)-2-quinazolinylguanidine,
6-chloro-8-methyl-4-(4-methylphenyl)-2-quinazolinylguanidine,
6-chloro-4-(4-fluorophenyl)-2-quinazolinylguanidine,
6-chloro-4-(2-chlorophenyl)-2-quinazolinylguanidine,
4-(3-methylphenyl)-2-quinazolinylguanidine,
6-chloro-4-(3-fluorophenyl)-2-quinazolinylguanidine,
6-chloro-8-chloro-4-phenyl-2-quinazolinylguanidine,
6-chloro-7-chloro-4-phenyl-2-quinazolinylguanidine,
and their physiologically acceptable salts and solvates.
